(19) United States
(12) Patent Application Publication
(10) Pub. No.: US 2020/0324041 A1
(43) Pub. Date: Oct. 15, 2020

(54) METHODS AND SYSTEMS ASSOCIATED A MEDICAL SLEEVE FOR INTRAVENOUS THERAPY LINES

(71) Applicant: Savage Lines, Del Rio, TX (US)

(72) Inventors: Lawrence Ervin, Del Rio, TX (US); Doug Craig, Del Rio, TX (US)

(21) Appl. No.: 16/783,794

(22) Filed: Feb. 6, 2020

Related U.S. Application Data
(63) Continuation of application No. 62/833,050, filed on Apr. 12, 2019.

(51) Int. Cl. A61M 5/14; A61M 39/10

(52) U.S. Cl. CPC A61M 5/1418; A61M 2209/08; A61M 2039/1066; A61M 39/10

(57) ABSTRACT

A medical sleeve for intravenous therapy (IV) lines. More particularly, systems that are configured to limit the tangling of IV lines or other types of cords, lines, cables, etc. positioned within the sleeve.

### BACKGROUND INFORMATION

### Field of the Disclosure

Examples of the present disclosure are related to systems and methods for a medical sleeve for intravenous therapy (IV) lines. More particularly, embodiments relate to systems that are configured to limit the tangling, synching, pulling, etc. of IV lines or other types of cords, lines, cables, etc. positioned within the sleeve.

### Background

IV is a therapy that delivers fluids directly into a vein. Utilizing IV lines medications and fluid replacements can be more efficiently and effectively delivered to the body. A standard IV infusions set includes a pre-filled container of fluids, with an attachment that allows the fluid to flow through lines. Conventional infusions may use multiple IV lines.

However, when in use, the multiple IV lines may become tangled, coupled together, cinched, etc. If the IV lines become tangled together there is a much higher likelihood that the IV lines may be pulled out of the patient or the container of fluid. When the IV lines become cinched, folded over, etc. the passageway through the IV lines may become blocked, resulting in the patient not receiving a correct amount of fluids. This likelihood of tangling, cinching, and/or pulling increases when the patient and the IV lines are being transported. Additionally, the likelihood of tangling, cinching, and being pulled increases while more of the IV lines are exposed.

Accordingly, needs exist for more effective and efficient methods and systems sleeves configured to secure IV lines in place, while allowing the IV lines to be folded and transported.

### SUMMARY

Embodiments of this disclosure are related to a medical device that is configured to secure IV lines in place, while allowing the IV lines to be transported between locations while reducing factors associated with tangling. The medical device may include a sleeve with a plurality of plates.

The sleeve may be a piece of non-rigid fabric, such as textiles, cloth, etc. The sleeve may include first coupling mechanisms positioned on and extending along a first edge of the sleeve, wherein the first edge extends along a longitudinal axis of the sleeve. The sleeve may also include second coupling mechanisms positioned on and extending along a second edge of the sleeve, wherein the second edge extends along the longitudinal axis. The first edge and the second edge of the sleeve may be configured to be coupled together via the first coupling mechanisms and the second coupling mechanisms along a central axis of the sleeve. In embodiments, the first coupling mechanisms and second coupling mechanisms may be corresponding hooks and loops, buttons, buckles, adhesives, etc.

The plates may be positioned along the central axis of the sleeve. In embodiments, there may be at least three plates, wherein the sleeve is configured to be folded across a lateral axis of the sleeve between a first plate and a second plate, and across a lateral axis of the sleeve between the second plate and a third plate. In embodiments, a first distance between the first plate and second plate may be different than a second distance between the second plate and the third plate. This may allow for compact folding of the sleeve without tangling of cinches embedded IV lines.

Each of the plates may include a plurality of hooks. The plurality of hooks may create grooves to receive the IV lines, wherein the grooves extend along the longitudinal axis of the sleeve. In embodiments, a first and second hook may have an opening that is facing a first direction, while a third hook may have an opening that is facing a second direction, wherein the first direction and the second direction are opposite directions. In embodiments, the second hook may be positioned between the first hook and third hook on a plate.

In embodiments, each of the plates and corresponding hooks may be aligned along the longitudinal axis of the sleeve when the sleeve is not folded. When the sleeve is folded, each of the plates may be misaligned along multiple planes, such as a transverse plane and a frontal plane, which may limit the cinching of the IV lines.

Further embodiments may also include an IV line holder. The IV line holder may be configured to be configured to be coupled with an IV line stand, pole, etc. to support the IV lines. The IV line holder may include a coupling orifice that is configured to be inserted onto the IV line pole, and a plurality of hooks. Each of the hooks may include a groove that forms a corresponding hook. The groove may form a passageway that is greater than one hundred eighty degrees, with an upwardly angled hook. This may limit the IV lines from exiting the passageway.

These, and other, aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions, or rearrangements.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 depicts a perspective view of a medical device, according to an embodiment.

FIG. 2 depicts a medical device, according to an embodiment.

FIG. 3 depicts a medical device, according to an embodiment.

FIG. 4 depicts a plate, according to an embodiment.

FIG. 5-7 depict various views of a medical device, according to an embodiment.

FIG. 8 illustrates a method for using a medical device, according to an embodiment.

FIGS. 9-12 depict a hanging device, according to an embodiment. FIG. 13-20 depict various views of a plate, according to an embodiment.

FIG. 21-27 depict various views of a plate, according to an embodiment.

Corresponding reference characters indicate corresponding components throughout several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

In the following description, numerous specific details are set forth in order to provide a thorough understanding of the present embodiments. It will be apparent to one having ordinary skill in the art, that the specific detail need not be employed to practice the present embodiments. In other instances, well-known materials or methods have not been described in detail in order to avoid obscuring the present embodiments.

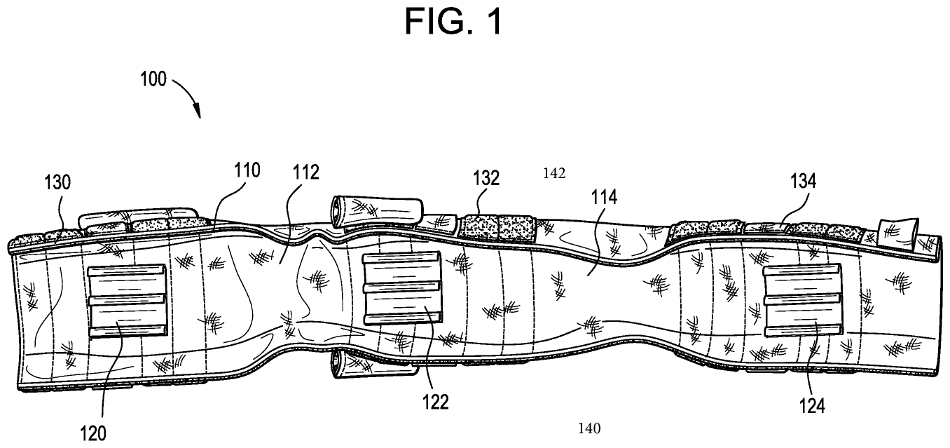

FIG. 1 depicts a perspective view of medical device 100, according to an embodiment. Medical device 100 may include a sleeve 110, plates 120, 122, 124, and coupling mechanisms 130, 132, 134.

Sleeve 110 may be formed of pliable, non-rigid fabrics, textiles, etc. The material associated with sleeve 110 may allow sleeve 110 to be folded over itself. Sleeve 110 may be configured to be folded over itself at multiple locations 112, 114, which may form non-uniform segments of sleeve 100. As such, a first distance from a first end of sleeve 110 to a first folding location 112, may be a different than a second distance from first folding location 112 to second folding location 114, which may also different than a third distance from second folding location 114 to a second end of sleeve 110.

In embodiments, a first edge of sleeve 110 extending from the first end of sleeve 110 to a second end of sleeve 110 may include a first coupling mechanism 140. A second edge of sleeve 110 extending from the first end of sleeve 110 to the second end of sleeve 110 may include a second coupling mechanism 142. The first coupling mechanism 140 may be positioned on an internal surface of the first edge, and the second coupling mechanism 142 may be positioned on an external surface of the second edge. In embodiments, the first coupling mechanism 140 may be configured to be aligned with the second coupling mechanism 142 along a central axis of sleeve 110 to selectively open and close a compartment within sleeve 110. In embodiments, first coupling mechanism 140 and second coupling mechanism 142 may be configured to be coupled together before sleeve 110 is folded at first folding location 112 and/or second folding location 114. Further, first coupling mechanism 140 and second coupling mechanism 142 may be any type of coupling mechanisms, such as hooks and loops, buttons, fasteners, zippers, etc.

Plates 120, 122, 124 may be configured to be secure IV lines in place along a longitudinal axis of sleeve 110 within the selectively opened and closed compartment within sleeve 110. Plates 120, 122, 124 may have multiple hooks that are positioned in different directions, which may limit the tangling of IV lines within sleeve 110. In embodiments, a distance between a first plate 120 and second plate 122 may be different than a distance between second plate 122 and third plate 124. This may allow sufficient space for folding locations 112, 124, which may be positioned between first plate 120 and second plate 122 as well as between second plate 122 and third plate 124, respectively. The relative positioning of the plates 120, 122, 124 may also allow the plates 120, 122, 124 to be misaligned when sleeve 110 is folded over itself, which may limit compression against the embedded IV lines.

Each of the plates 120, 122, 124 may have a corresponding coupling mechanism 130, 132, 134, respectively. In embodiments, responsive to folding sleeve 110 at second folding location 114, coupling mechanism 134 may be positioned on top of and over coupling mechanism 132. Then, sleeve 110 may be folded at first folding location 112, and coupling mechanism 130 may be position on top of an over coupling mechanism 134. The coupling mechanisms 130, 132, 134 may be any type of coupling mechanism, buttons, snaps, hooks and loops, zippers, etc.

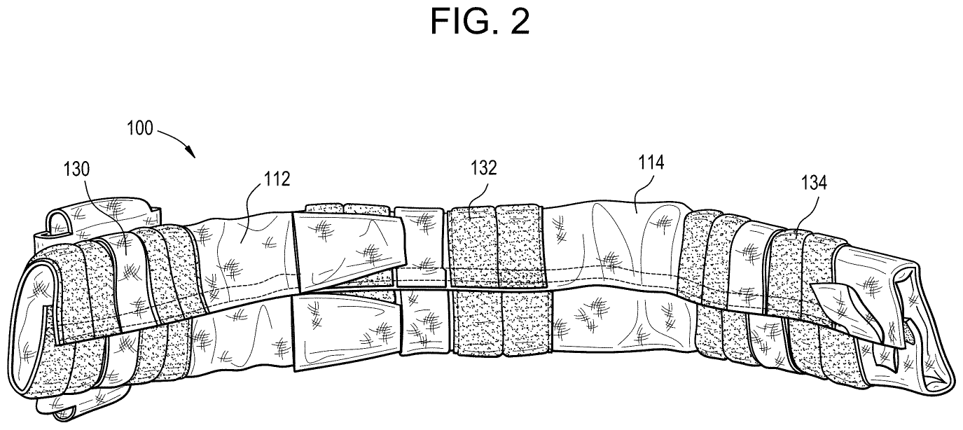

FIG. 2 depicts medical device 100, according to an embodiment. Elements depicted in FIG. 2 may be described above, and for the sake of brevity a further description of these elements may be omitted.

As depicted in FIG. 2, coupling mechanisms 140 and 142 may be coupled together along a central axis of sleeve 110. This may enable the compartment within sleeve 110 to be protected, and not exposed. This may limit the exposure of IV lines secured within plats 120, 122, 124.

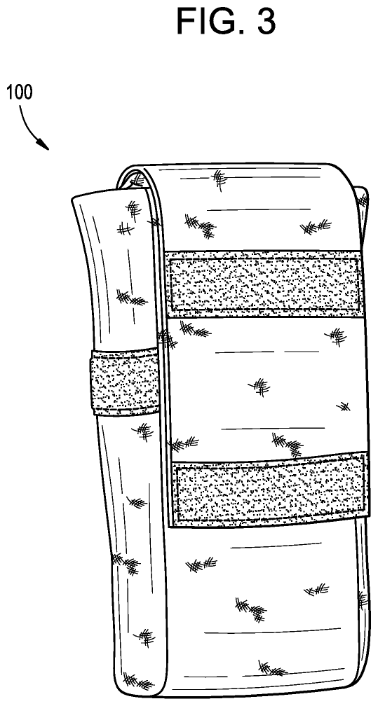

FIG. 3 depicts medical device 100, according to an embodiment. Elements depicted in FIG. 3 may be described above, and for the sake of brevity a further description of these elements may be omitted.

As depicted in FIG. 3, sleeve 110 may be folded over a lateral axis of sleeve 110 at multiple folding points 112, 114. This may allow sleeve 110 to be compact while being transported.

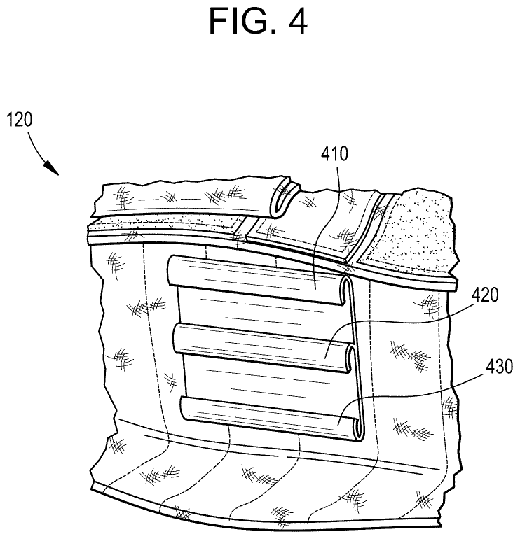

FIG. 4 depicts a plate 120, according to an embodiment. Elements depicted in FIG. 4 may be described above, and for the sake of brevity a further description of these elements may be omitted.

Each of the plates may include a plurality of hooks 410, 420, 430. The plurality of hooks may create grooves to receive IV lines. The grooves may be created between an end of the hook 410, 420, 430 and an upper surface of the plates. The grooves may extend along the longitudinal axis of the sleeve 110. In embodiments, a first hook 410 and second hook 420 may have an opening that is facing a first direction, while a third hook 430 may have an opening that is facing a second direction, wherein the first direction and the second direction are opposite directions. In embodiments, the second hook 420 may be positioned between the first hook 410 and third hook 430 on a plate 120.

In embodiments, when the sleeve is elongated, and not folded over itself, each of the hooks 410, 420, 430 on the different plates may be aligned along the longitudinal axis of the sleeve 110. This may allow IV lines disposed within sleeve 110 to extend in parallel to a longitudinal axis of the sleeve 110.

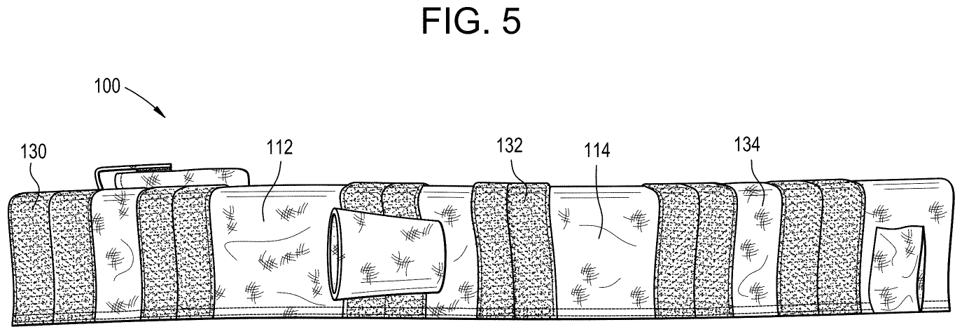

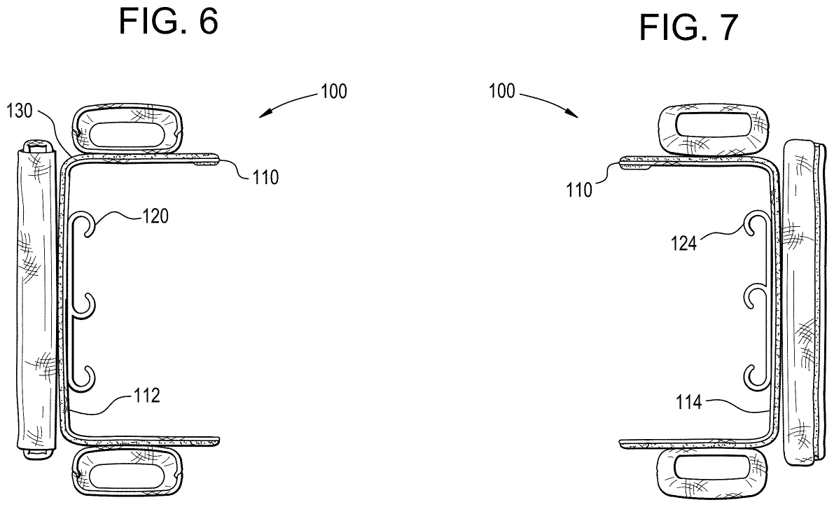

FIG. 5-7 depict various views of a medical device 100, according to an embodiment. Elements depicted in FIG. 5-7 may be described above, and for the sake of brevity a further description of these elements may be omitted.

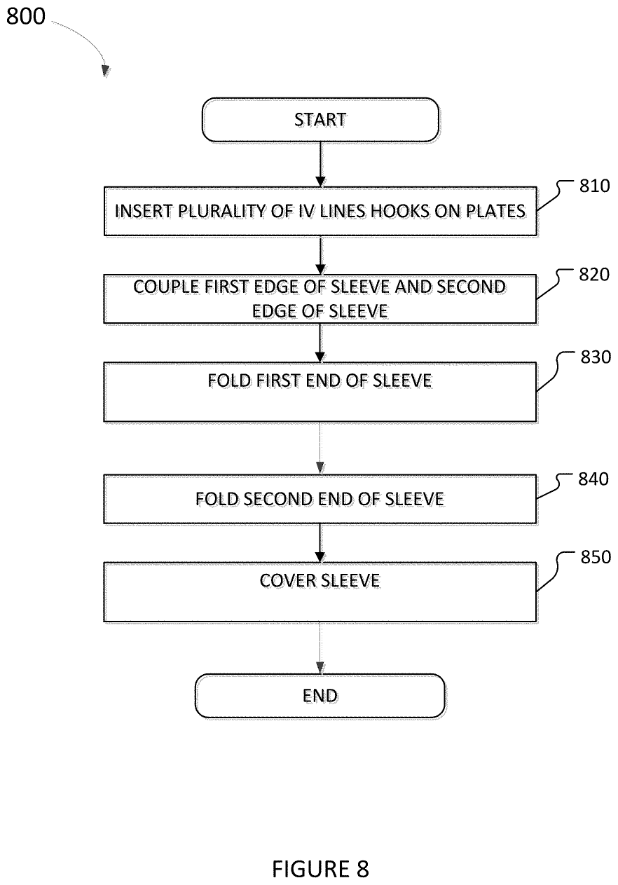

FIG. 8 illustrates a method 800 for using a medical device, according to an embodiment. The operations of method 800 presented below are intended to be illustrative. In some embodiments, method 800 may be accomplished with one or more additional operations not described, and/or without one or more of the operations discussed. Additionally, the order in which the operations of method 800 are illustrated in FIG. 8 and described below is not intended to be limiting.

At operation 810, a plurality of IV lines may be inserted through a plurality of axially aligned hooks on plates positioned on a sleeve.

At operation 820, first coupling mechanisms on a first edge of the sleeve may be coupled with second coupling mechanisms on a second edge of the sleeve along a central axis of the sleeve. Responsive to coupling the first coupling mechanisms with the second coupling mechanisms, the IV lines and the plurality of hooks may be covered and encompassed by the sleeve.

At operation 830, a first end of the sleeve may be folded at a first folding location positioned between a second plate and a third plate.

At operation 840, a second end of the sleeve may be folded at a second folding location positioned between a first plate and the second plate, wherein a first distance between the first folding location and the first end of the sleeve may be different than a second distance between the second end of the sleeve and the second folding location.

At operation 850, the sleeve with the covered IV lines may be transported from a first location to a second location.

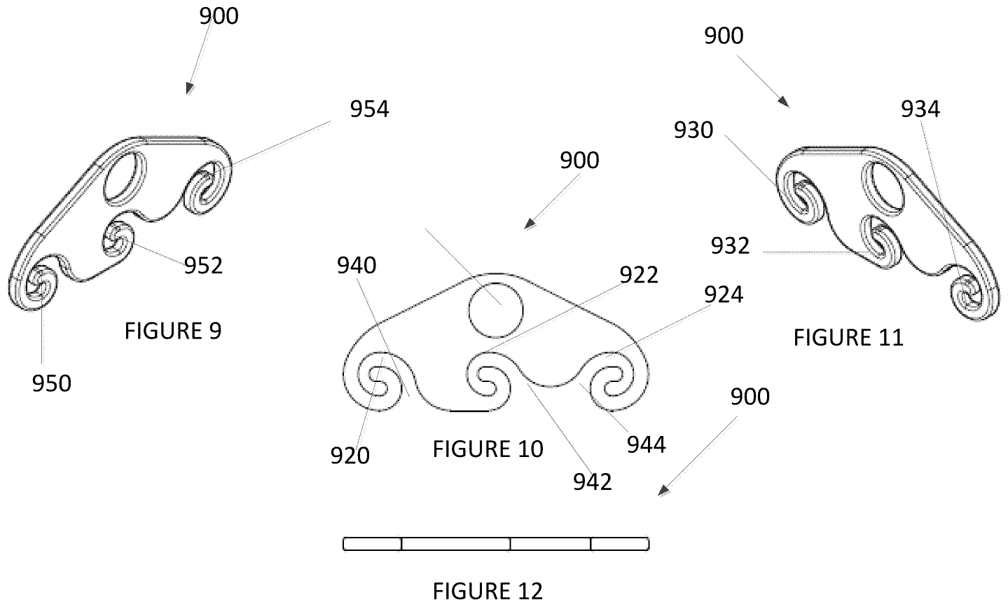

FIGS. 9-12 depict a hanging device 900, according to an embodiment. Hanging device 900 may be configured to operate with an IV line stand to organize connected IV lines.

As depicted in FIGS. 9-12, hanging device 900 may include a hanging orifice 910, a plurality of grooves 920, 922, 924 forming a plurality of corresponding hooks 930, 932, 934. Hanging orifice 910 may be a hole, opening, etc. extending from a front surface of hanging device 900 to the rear surface of hanging device 900, and may be configured to be positioned around a pole, shaft, hook, peg, etc. associated with an IV line holder.

Each of the plurality of grooves 920, 922, 924 may have a corresponding opening 940, 942, 944 and an end 950, 952, 954. Each of the openings 940, 942, 944 may face away from a corresponding end 950, 952, 954, wherein the corresponding groove makes a turn between one hundred eighty degrees and three hundred degrees to form a corresponding hook 930, 932, 934.

The rotation of the grooves 920, 922, 924 to form the corresponding hooks 930, 932, 934 may limit that possibility of an IV line slipping, sliding, etc. while embedded within a hook.

Furthermore, the outermost grooves 920 and 924 may face opposite directions. This may enable positioned IV lines in different orientations to further reduce slipping, sliding, etc.

FIG. 13-20 depict various views of a plate 120, according to an embodiment. Elements depicted in FIGS. 13-20 may be described above, and for the sake of brevity a further description of these elements may be omitted.

Specifically, FIGS. 13 and 20 depict perspective views of plate 120, FIGS. 14 and 19 depict side views of plate 120, FIG. 16 depicts a top view of plate 120, FIG. 17 depicts a front view of plate 120, and FIG. 18 depicts a back view of plate 120.

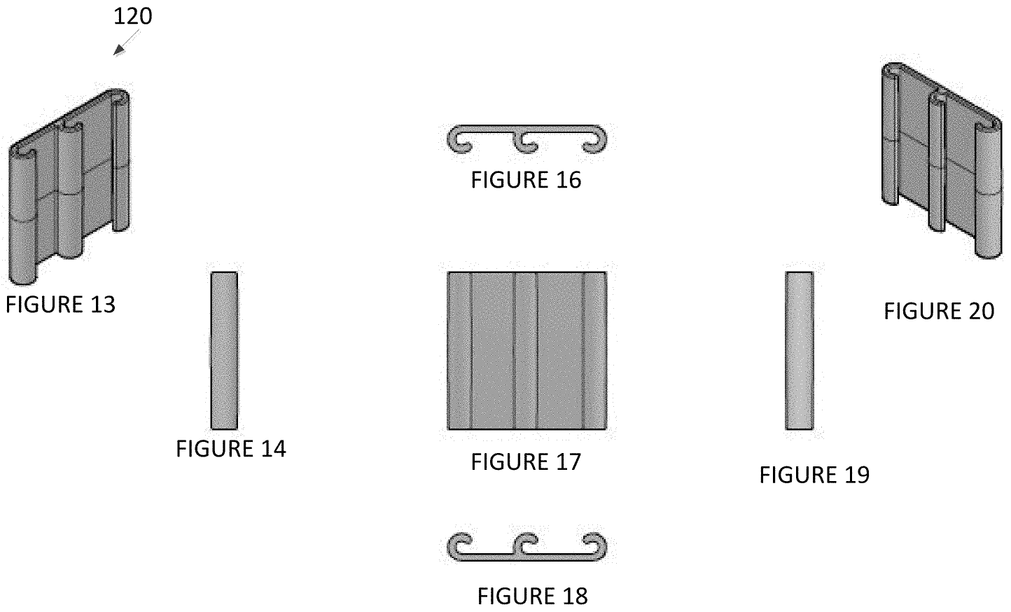

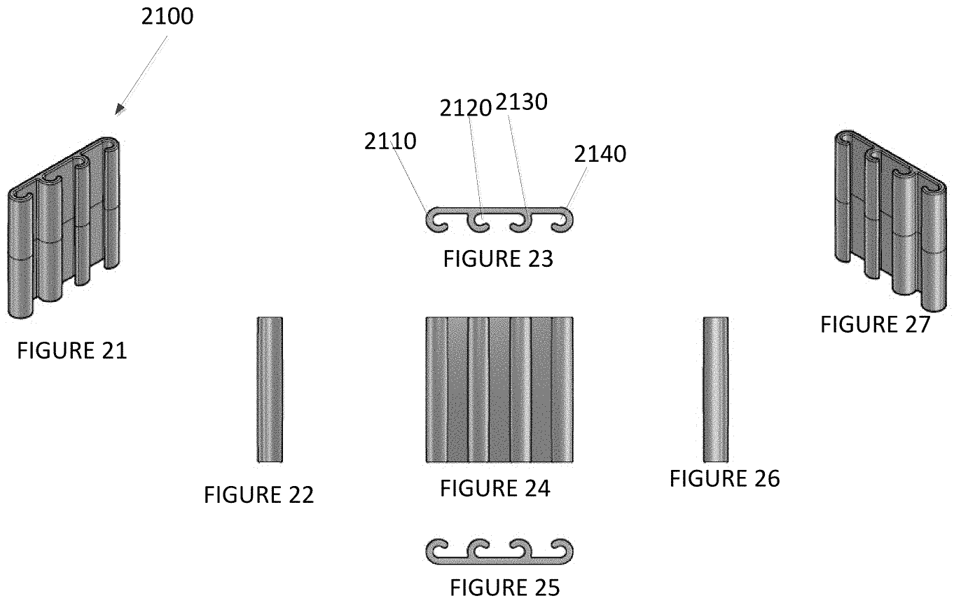

FIG. 21-27 depict various views of a plate 2100, according to an embodiment. Elements depicted in FIGS. 21-27 may be described above, and for the sake of brevity a further description of these elements may be omitted.

Specifically, FIGS. 21 and 27 depict perspective views of plate 2100, FIGS. 22 and 26 depict side views of plate 2100, FIG. 23 depicts a top view of plate 2100, FIG. 24 depicts a front view of plate 2100, and FIG. 25 depicts a back view of plate 2100. As depicted in FIGS. 21-27, plate 2100 may include a plurality of grooves 2110, 2120, 2130, 2140. The grooves on a first side of a central axis of plate 2100 may be configured to face a first direction, and the grooves on a second side of the central axis of plate 2100 may be configured to face a second direction. This may reduce the likelihood of IV lines becoming tangled.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Reference throughout this specification to “one embodiment”, “an embodiment”, “one example” or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

1. A medical device comprising:
a sleeve formed of fabric with a first edge and a second edge, wherein the first edge and the second edge extend from a first end to a second end of the sleeve;
a first coupling mechanism positioned along the first edge from the first end to the second end;
a second coupling mechanism positioned along the second edge from the first end to the second end, the first coupling mechanism and the second coupling mechanism being configured to be selectively coupled together along a central axis of the sleeve;
a plurality of plates positioned along the central axis of the sleeve, each of the plates including a plurality of hooks configured to secure IV lines in place, wherein corresponding hooks on a first plate and a second plate are aligned along a longitudinal axis of the sleeve, the plurality of plates including the first plate, the second plate and a third plate, wherein a first length from the first plate to the second plate is different from a second length from the second plate to the third plate, a width of the sleeve is larger than that of the first plate, second plate, and third plate, wherein the plurality of hooks include a first hook, a second hook, and a third hook, the first hook and the second hook face a same direction towards the first edge of the sleeve, and the third hook that faces a different direction towards the second edge of the sleeve, each of the first hook, the second hook, and the third hook extending along an entirety of a longitudinal axis of a corresponding plate;
a first folding location extending across a lateral axis of the sleeve, the lateral axis being perpendicular to the central axis of the sleeve, the first folding location being positioned between the first plate and the second plate, wherein when the sleeve is folded at the first folding location a proximal end of the sleeve is positioned adjacent to and encompassed by the first folding location;
a second folding location extending across the lateral axis of the sleeve, a first distance from the first end of the sleeve to the first folding location being different than a second distance from the second end of the sleeve to the second folding location, the second folding location being positioned between the second plate and the third plate, wherein when the sleeve is folded at the second folding location.

2. (canceled)

3. The medical device of claim 1, wherein the first coupling mechanism and second coupling mechanism are hooks and loops.

4. (canceled)

5. The medical device of claim 1, wherein the first hook on each of the plurality of plates is positioned along a first longitudinal axis along the sleeve, the second hook on each of the plurality of plates is positioned along a second longitudinal axis along the sleeve, and the third hook on each of the plurality of plates is positioned along a third longitudinal axis along the sleeve.

6. The medical device of claim 5, wherein a first IV line is configured to be positioned on the first hook on each of the plurality of plates positioned along the first longitudinal axis.

7. The medical device of claim 1, further comprising:
a holding device configured to hold a plurality of IV lines.

8. The medical device of claim 1, wherein the holding device includes a plurality of groove, each of the plurality of grooves being configured to secure an independent IV line.

9. The medical device of claim 1, wherein each of the plurality of grooves has a curvature of at least one hundred eighty degrees.

10. The medical device of claim 1, wherein at least two of the plurality of grooves face opposite directions.
